(19) United States
(12) Patent Application Publication
(10) Pub. No.: US 2026/0241635 A1
(43) Pub. Date: Aug. 20, 2026

(54) 3D BIOPRINTING OF STRUCTURE CELL AGGREGATES AND ORGANOIDS

(71) Applicant: CARNEGIE MELLON UNIVERSITY, Pittsburgh, PA (US)

(72) Inventors: Adam Walter FEINBERG, Pittsburgh, PA (US); Samuel Patrick MOSS, Pittsburgh, PA (US)

(21) Appl. No.: 18/992,644

(22) PCT Filed: Oct. 13, 2023
(86) PCT No.: PCT/US2023/035131

§ 371 Date: Jan. 9, 2025

Related U.S. Application Data
(60) Provisional application No. 63/416,075, filed on Oct. 14, 2022.

(51) Int. Cl. B29C 64/118; B29C 64/209; B29C 64/336; B29C 64/386; B29C 64/40; B29C 71/02; B29K 105/00; B29L 31/00; B33Y 10/00; B33Y 30/00; B33Y 40/20; B33Y 50/00; B33Y 70/10; B33Y 80/00; C12N 5/077

(52) U.S. Cl. CPC B29C 64/118; B29C 64/209; B29C 64/336; B29C 64/386; B29C 64/40; B29C 71/02; B33Y 10/00; B33Y 30/00; B33Y 40/20; B33Y 50/00; B33Y 70/10; B33Y 80/00; C12N 5/0658; B29K 2089/00; B29K 2105/0058; B29L 2031/753; C12N 2513/00; C12N 2533/50; C12N 2533/54; C12N 2533/56; C12N 2533/70

(57) ABSTRACT

Additive manufacturing methods, additive manufacturing systems, and products thereof are provided. The method comprises depositing a bioink into a support material based on a first computer model of an object, thereby forming a first portion of an object in the support material. The bioink comprises cells. The method comprises depositing a structure material into the support material based on the first computer model of an object, thereby forming a first portion of a scaffold for the object. The structure material is different from the bioink and the structure material comprises a polymer. The method comprises repeating the depositing of the bioink and the structure material as necessary to additively form the object and the scaffold within the object.

### RELATED APPLICATIONS

The present application claims priority to U.S. provisional patent application Ser. No. 63/416,075, filed Oct. 14, 2022, which is incorporated herein by reference in its entirety.

### BACKGROUND

Recently, 3-dimensional (3D) bio-printing has emerged as a viable platform for engineering tissues, with exciting applications as platforms for drug discovery and disease modeling as well as new therapies for tissue regeneration. However, translation of these technologies from the laboratory into industry and the clinic has been challenging.

### SUMMARY

In one general aspect, the present disclosure is related to an additive manufacturing method. The method comprises depositing a bioink into a support material based on a first computer model of an object and a scaffold for the object, thereby forming a first portion of an object in the support material. The bioink comprises cells and optionally a bio compatible polymer (e.g., fibrinogen) and water. In various examples, the support material can comprise a hydrogel and optionally comprises thrombin. The method comprises depositing a structure material into the support material based on the first computer model, thereby forming a first portion of the scaffold. In various examples, the scaffold can be configured to guide the cell-mediated compaction of the object. The structure material is different from the bioink and the structure material comprises a polymer. In various examples, the polymer comprises a collagen material and the structure material is acellular. The method comprises repeating the depositing of the bioink and the structure material as necessary to additively form the object and the scaffold within the object. In certain examples, the method comprises culturing the object to form a cell aggregate.

In another general aspect, the present disclosure is related to an additive manufacturing system. The system comprises an extruder assembly, a material deposition region, and a processor. The extruder assembly comprises a first nozzle and a second nozzle. The first nozzle is configured to deposit, according to a first computer model, bioink into a support material to additively form an object made of the bioink in the support material. The second nozzle is configured to deposit, according to the first computer model, structure material into a support material to additively form a scaffold made of the structure material in the support material. The material deposition region is configured to hold the support material. The processor is in communication with the extruder assembly. The processor is programmed to perform a method for additive manufacturing as described herein.

A product can be fabricated by the method for additive manufacturing and/or the system for additive manufacturing described herein.

Various embodiments and implementations of the present invention provide many benefits and improvements relative to prior additive printing techniques, such as, for example, techniques related to embedded printing. For example, providing a scaffold can guide the compaction of the object to a desired shape, cellular organization, pattern of cellular signaling, tissue structure development, and/or physiological function. These and other benefits that are potentially realizable through various implementations of the present invention will be apparent from the description that follows.

It is understood that the inventions described in this specification are not limited to the examples summarized in this Summary. Various other aspects are described and exemplified herein.

### BRIEF DESCRIPTION OF THE DRAWINGS

The features and advantages of the examples, and the manner of attaining them, will become more apparent, and the examples will be better understood, by reference to the following description taken in conjunction with the accompanying drawing, wherein:

FIG. 1 is a block diagram of an example of an additive manufacturing FRE system according to the present disclosure, the X-axis is coming out of the page.

FIG. 2 is flow chart of an example of an additive manufacturing FRE method according to the present disclosure.

FIG. 3 is a diagram illustrating compaction of the object around a scaffold according to the present disclosure.

FIG. 4 is a computer model of an object.

FIG. 5 is a graph illustrating a mean average diameter of objects printed with bioinks having varying fibrinogen concentrations during compaction.

FIG. 6 is micrographs of objects printed with bioinks having varying fibrinogen concentrations during compaction.

FIG. 7 is a graph illustrating a mean average diameter of objects printed with bioinks having varying cell concentrations during compaction.

FIGS. 8A-8B is micrographs of objects printed with bioinks having varying cell concentrations during compaction.

FIG. 9 is micrographs of objects printed with bioinks including and not including Xanthan Gum.

FIG. 10 is micrographs of objects printed at various size scales during compaction.

FIG. 11 is a graph illustrating a mean average diameter of objects with varying size scales during compaction.

FIG. 12 is a computer model and images of an object and a scaffold formed as an open square according to the present disclosure.

FIG. 13 is a computer model and images of an object and a scaffold formed as an enclosed square according to the present disclosure.

FIG. 14 is a computer model and images of an object and a scaffold formed as an enclosed circle according to the present disclosure.

FIG. 15 is a computer model and images of an object and a scaffold formed as an enclosed rectangle according to the present disclosure.

FIG. 16 is a computer model and images of an object and a scaffold formed as an 2.5 mm×1.05 mm rectangle according to the present disclosure.

FIG. 17 is a computer model and images of an object and a scaffold formed as an 3.0 mm×1.05 mm rectangle according to the present disclosure.

FIG. 18 is a computer model and images of an object and a scaffold formed as an 3.5 mm×1.05 mm rectangle according to the present disclosure.

FIG. 19 is a computer model and images of an object and a scaffold formed as an 2.5 mm×1.50 mm rectangle according to the present disclosure.

FIG. 20 is a diagram illustrating compaction of the object around two scaffolds according to the present disclosure.

FIG. 21 is a cross-sectional view of the object and two scaffold in FIG. 20 prior to compaction.

The exemplifications set out herein illustrate certain embodiments, in one form, and such exemplifications are not to be construed as limiting the scope of the appended claims in any manner.

### DETAILED DESCRIPTION

As used herein, “additive manufacturing” means a process of joining materials to make objects from 3D model data, usually layer upon layer, as opposed to subtractive manufacturing methodologies. For example, additive manufacturing can comprise fused deposition modeling (FDM) and Freeform Reversible Embedding (FRE), among other technologies. FDM can comprise extruding a material by heating it to a temperature above its melting temperature and depositing the extruded material in a pattern to form a layer of an object. Subsequent layers can be deposited on top of the previous layer as necessary to form an object.

FRE is similar to FDM, but instead of depositing a material on top of previous depositions or supports, FRE embeds a print material near other embedded deposits inside a support material and relies on the triggered assembly or reorganization of the material using targeted heating, photopolymerization, crosslinking, slow reaction kinetics, application of binders, and/or other curing technique. For example, the support material may provide divalent cations for ionic crosslinking, such that when the print material contacts the support material, the printed material can begin to cure. In certain examples, the print material may not cure and can hold its shape based on a thixotropic and/or yield-stress property.

For additive manufacturing techniques such as FDM, support materials are usually as stiff as the print material, printed as part of the previous layer, and placed only underneath or neighboring the print layers to prevent deformations. In FRE, the support material can surround the extrusion nozzle and the print material can be deposited inside the support material. The support material can allow for deposition of various materials while maintaining a buoyant, physical support for already embedded deposits of print material. When two embedded deposits of print material with a predetermined distance inside of the support material, they can fuse. After printing, the support material can be removed from the deposited print material to form a fully assembled object from the deposited print material.

In FRE, an object can be printed in any direction in 3D space and is not limited to layer-by-layer printing. For example, a structure can also be printed layer by layer in an X-Y plane, or a non-X-Y plane, such as the X-Z plane, or in a plane at any angle offset from the X-Y Plane. An object can also be printed utilizing FRE in a non-planar fashion, such as, for example, in a curved path such as a helix. Utilizing FRE can enable printing of objects with mechanical properties that are different in the plane of printing versus orthogonal to the plane of printing or other angle to the plane of printing. Additional details regarding the FRE process can be found in U.S. Pat. No. 10,150,258, titled ADDITIVE MANUFACTURING OF EMBEDDED MATERIALS, filed Jan. 29, 2016, U.S. patent application Ser. No. 17/754,115, titled MODIFICATION OF RHEOLOGY AND MACHINE PATHING FOR IMPROVED 3D PRINTING OF SOFT MATERIALS, and U.S. patent application Ser. No. 18/246,225, titled TRANSPARENT SUPPORT BATH FOR EMBEDDED 3D PRINTING AND SYSTEM FOR IN PROCESS MONITORING, filed Mar. 22, 2023, each of which are hereby incorporated by reference herein.

As the demand for donor tissue and organs continues to outpace the supply, clinicians are turning to regenerative medicine and tissue engineering strategies to create tissue de novo. 3D bioprinting using FRE has emerged as a way to build these tissues using robotic control to precisely pattern cells and biological hydrogels. However, this technology has been slowed by the difficulty of printing these soft, deformable materials into complex 3D architectures that recapitulate anatomic structure from the micro to macro length scale, and by a lack of ability to control the growth of the patterned cells after printing. Given the structure-function relationships in most tissue types, establishing that the 3D architecture of a bioprinted construct (e.g., object) matches its intended anatomic design can be desirable.

The present disclosure provides methods, systems, and materials that can enhance process reliability during the FRE process, other 3D bio-printing process, or other additive manufacturing process and enable desirable growth of cells to a suitable size and shape after printing. For example, the present disclosure provides an additive manufacturing method and an additive manufacturing system. The method comprises depositing a bioink into a support material based on a first computer model of an object and a scaffold for the object, thereby forming a first portion of an object in the support material. The method comprises depositing a structure material into the support material based on the first computer model, thereby forming a first portion of the scaffold. The structure material is different from the bioink and the structure material comprises a polymer. The method comprises repeating the depositing of the bioink and the structure material as necessary to additively form the object and the scaffold within the object.

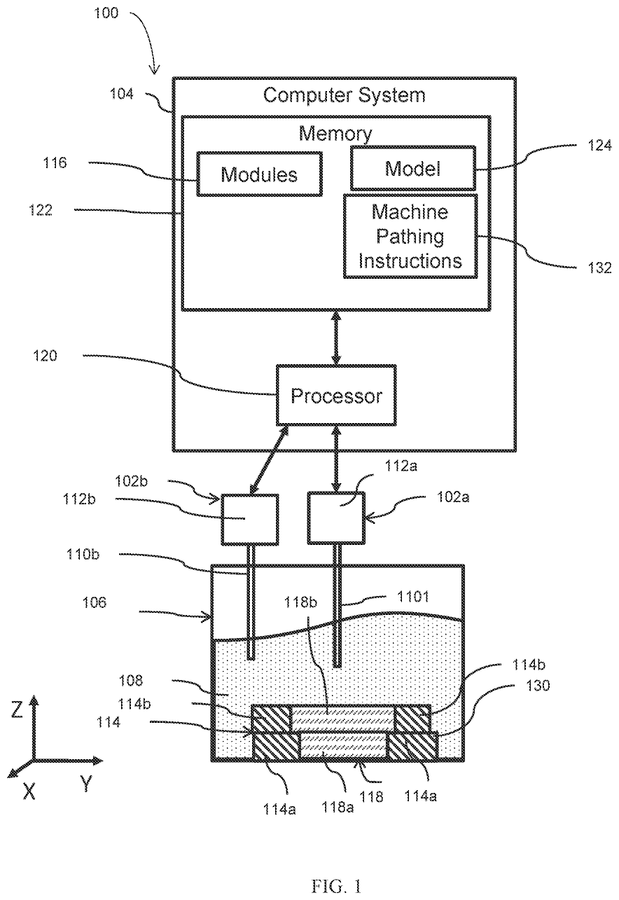

Referring to FIG. 1, a block diagram illustrating an example of an additive manufacturing system 100 for FRE according to the present disclosure is provided. The system 100 comprises an extruder assembly 102a, a computer system 104, a material deposition region 106, optionally an extruder assembly 102b. In various examples, additional extruders, additional nozzles, and/or a detector may be added to the additive manufacturing system to increase the printing capabilities of the additive manufacturing system. The computer system 104 can be in signal/data communication with the extruder assembly 102a and the extruder assembly 102b (such as via a wired and/or wireless data bus or link). The computer system 104 can be configured through programming to control the operation of the extruder assembly 102a and the extruder assembly 102b. The computer system 104 can also receive data from and send data (e.g. control data) to the extruder assemblies 102a, 102b. The components may be in communication with the computer system 104 via any suitable type of data bus (e.g., parallel or bit serial connections).

Each extruder assembly 102a and 102b may be a syringe-based extruder, which can include a reservoir 112a and 112b, respectively, (e.g., a barrel of a syringe) for receiving and storing structure material or support material, and a nozzle 110a and 110b, respectively, (e.g., a needle) which can be in fluid communication with the respective reservoir 112a or 112b and can receive the structure material or the bioink material from the reservoir 112a or 112b. For example, the reservoir 112a can comprise structure material and the structure material can be extruded through the nozzle 110a and the nozzle 110a can be configured to deposit the extruded structure material in the support material 108 disposed in the material deposition region 106. The reservoir 112b can comprise bioink and the bioink can be extruded through the nozzle 110b and the nozzle 110b can be configured to deposit the extruded support material in the material deposition region 106. In various examples, a single extruder assembly may be present that includes valving to print either the bioink, the structure material through, or an additional material a single nozzle or different nozzles, when desired.

In various examples, the extruder assembly 102a, the extruder assembly 102b and/or additional components can comprise a gantry or other robotic device to support and/or move the extruder assembly 102a and/or the extruder assembly 102b relative to the material deposition region 106. Optionally, the extruder assembly 102a and/or the extruder assembly 102b can comprise a motor assembly or other movement assembly configured to translate and/or rotate the gantry and/or robotic device. In various examples, each extruder assembly 102a and 102b comprises an actuator (e.g., a motor) configured to depress a plunger into the respective reservoir 112a or 112b to extrude material through the nozzle 110a or 110b into material deposition region 106 as nozzle 110a or 110b is translated through the material deposition region 106 to additively form an object 114.

The computer system 104 comprises one or more processors 120 operatively coupled to one or more non-transitory memories 122 (only one processor 120 and one memory 122 are shown in FIG. 1 for simplicity). The processor 120 may comprise one or multiple processing cores. The memory 122 can comprise primary storage (e.g., main memory that is directly accessible by the processor 120, such as RAM, ROM processor registers or processor cache); secondary storage (e.g., SSDs or HDDs that are not directly accessible by the processor); and/or off-line storage. The memory 122 stores computer instructions (e.g., software) that are executed by the processor 120. The processor 120 can be configured (through execution of the software stored in the memory 122) to control operation of the extruder assemblies 102a, 102b to thereby control the deposition of the structure material and bioink through the nozzles 110a, 110b. For example, the processor 120 can control the flow rate of material through the nozzle 110a and/or 110b (e.g., by the actuation rate of a plunger in the respective extruder assembly 102a or 102b) and/or the pose of the extruder assembly 102a and the extruder assembly 102b relative to the material deposition region 106.

The memory 122 can store a digital or electronic computer model 124 of the object 114 and scaffold 118 for the object 114 (collectively assembly) to be manufactured by the additive manufacturing process. The computer model 124 can be loaded locally into the memory 122 or can be downloaded from another device (e.g., another computer device, cloud) that is in data communication with the computer system 104. To that end, the computer system 104 may comprise a network interface controller (NIC) (not shown) that connects the computer system 104 to a computer network. The computer model 124 can be in a variety of different digital or electronic formats, such as an STL file, a OBJ file, a FBS file, a COLLADA file, a 3DS file, an IGES file, a STEP file, a VRML/X3D file, a point cloud, or another 3D model file format type. The computer model 124 can be generated from image data of a biological structure, an engineered structure, a computationally derived structure, or a combination thereof. In various examples, the computer model 124 can be machine path instructions (e.g., G-code instructions), that may be directly input by an operator or can be downloaded from another device that is in data communication with the computer system 104.

The nozzle 110a can be configured to deposit a structure material into the support material 108 by applying a force to the structure material in the reservoir 112a such that the structure material can flow from the reservoir 112a through the nozzle 110a. The structure material can comprise a yield stress, a thixotropic property, an increased viscosity, or a combination thereof. In examples where the structure material comprises a yield stress, the force applied can be at least the yield stress. In certain examples, applying the force to the structure material can cause the structure material to flow through the nozzle. For example, with an increase viscosity, the force can overcome the increased viscosity and cause the material to flow through the nozzle. In examples wherein the structure material comprises a thixotropic property, the thixotropic property can cause the time scale to start flow of the structure material to be longer than the printing process.

The nozzle 110b can be configured to deposit a bioink into the material deposition region 106 by applying a force to the bioink in the reservoir 112b such that the bioink can flow from the reservoir 112b through the nozzle 110b.

In various examples, a plunger can be translated through the reservoir 112a and/or the reservoir 112b. In various examples, the force can be pneumatically applied or the deposition can be controlled by a progressive cavity pump. The application of the force can cause the material in the respective reservoir 112a, 112b to change form a solid or semi-solid state into fluid state (e.g., liquid), so that the material can be deposited into the material deposition region 106. The structure material and the bioink can be suspended in the support material 108 at a location where the respective structure material or bioink was deposited by the respective nozzle 110a, 110b within the support material 108. Since the processor 120 can control the extruder assemblies 102a, 102b and nozzles 110a, 110b, the deposition of material by the respective nozzle 110a, 110b can be based on the machine path instructions 132 as executed by the processor 120.

The extruder assembly 102a can move the nozzle 110a in two-dimensions when depositing structure material similar to FDM or in three-dimensions when depositing material, i.e., simultaneously in the X, Y, and Z directions. Further, the extruder assembly 102a, nozzle 110a, and/or material deposition region 106 can be rotatable. The machine pathing instructions 132 can be defined according to both Cartesian and polar coordinates, which can allow for the production of objects having complex geometries or very specific mechanical properties. 3D movement of the nozzle 110a during deposition of the structure material can enable, for example, additive manufacture of a helical spring in one constant motion. In various examples, other complex geometries are achievable with robotic arm assemblies capable of simultaneously controlling movement with six degrees of freedom (i.e., in any Cartesian or rotational direction). The extruder assembly 102b can move in the same or similar manner to extruder assembly 102a.

The depositing of the structure material and bioink can be repeated as necessary to additively form an object. For example, the processor 120 can control the nozzle 110a to deposit the structure material in portions (e.g., layers) in order to additively form the scaffold 118 in the support material 108 based on the computer model 124, another plane, and/or non-planar movement. As illustrated, the structure material was deposited in portion 118a and portion 118b of scaffold 118.

The processor 120 can control the nozzle 110b to deposit the bioink in portions (e.g., layers) in order to additively form the object in the support material 108 based on the computer model 124, another plane, and/or non-planar movement. As illustrated, the bioink was deposited in portion 114a and portion 114b to form the object 114.

The portions 114a, 114b, 118a, 118b, can be deposited in various sequences as desired. For example, portions 114a and 118a can be deposited prior to portions 114b and 118b. Portion 118a may not be partially and/or fully cured prior to deposition of portion 118b. The processor 120 can control the nozzles 110a 110b to deposit portions 114b, 118b, proximal to (e.g., adjacent, in contact with, directly on top of) the portions 114a, 118b, respectively, such that the deposition of the portion 114b contacts the portion 114a and the deposition of portion 118b contacts the portion 118a.

In various examples, the deposition of the structure material, bioink, and additional materials can occur in various stages. For example, a portion 118a of scaffold 118 can be formed by depositing structure material by the extruder assembly 102a and then the portion 114a of object 114 can be deposited by the extruder assembly 102a. In various examples, the bioink can be deposited prior to the structure material. In certain examples, the scaffold 118 may be completely formed prior to deposition of the object 114 or the object 114 may be completely formed prior to deposition of the scaffold 118.

The bioink can comprise cells. For example, the cells can comprise eukaryotic cells derived from an animal. The cells can be obtained from embryonic stem cells (ESCs), induced pluripotent stem cells (iPSCs), primary tissues, a cell line, or a combination thereof. The bioink can comprise at least 10 million cells per milliliter (mL) of bioink, such as, for example, at least 20 million cells per mL of bioink, at least 25 million cells per mL of bioink, at least 30 million cells per mL of bioink, at least 40 million cells per mL of bioink, at least 50 million cells per mL of bioink, at least 75 million cells per mL of bioink, or at least 100 million cells per mL of bioink. In various examples, the bioink can comprise a concentration of cells in a range of 25 million cells per mL of bioink to 1,000 million cells per mL of bioink or a range of 75 million cells per mL of bioink to 500 million cells per mL of bioink.

The bioink can comprise a bio compatible polymer, water, and optionally an additive. The bio compatible polymer can comprise a collagen material, an alginate material, a decellularized extracellular matrix material, a fibrinogen material, a fibrin material, a hyaluronic acid material, a protein material, a polysaccharide hydrogel material, a synthetic hydrogel material, a Matrigel, or a combination thereof. The protein material can comprise fibrinogen. The bioink can comprise at least 1 milligrams (mg) of fibrinogen per mL of bioink, such as, for example, at least 10 mg of fibrinogen per mL of bioink, at least 20 mg of fibrinogen per mL of bioink, at least 50 mg of fibrinogen per mL of bioink, at least 55 mg of fibrinogen per mL of bioink, at least 60 mg of fibrinogen per mL of bioink, or at least 70 mg of fibrinogen per mL of bioink. In various examples, the bioink can comprise a concentration of fibrinogen in a range of 1 mg of fibrinogen per mL of bioink to 1,000 mg of fibrinogen per mL of bioink, such as, for example, a range of 50 mg of fibrinogen per mL of bioink to 500 mg of fibrinogen per mL of bioink. The additive can comprise a rheological modifier (e.g., a high molecular weight polysaccharide such as, for example, xanthan gum, dextran, or hyaluronic acid) and/or a chelating agent (e.g., ethylenediaminetetraacetic acid (EDTA)). The bioink can comprise a concentration of the rheological modifier in a range of 0.1 weight percent to 2 weight percent by total weight of the bioink. The bioink can comprise a concentration of the chelating agent in a range of 1 mM to 20 mM, such as, for example, 1 mM to 10 mM.

The structure material can differ from the bioink. The structure material can be acellular (e.g., may not comprise cells) and/or the structure material can differ from the bioink by at least one mechanical property, such as, for example, a yield strength, a stiffness, a tensile strength, or a combination thereof.

The structure material can comprise a yield stress material that transitions between a fluid (e.g., liquid) state to a solid or semi-solid state by application of a pressure. For example, the structure material can be in a solid or semi-solid state in the extruder assembly 102a, a pressure can be applied to the structure material to transition the structure material to a fluid state such that the structure material can flow through the nozzle 110a and can be deposited into the support material 108. After leaving the nozzle 110a, the applied pressure to the structure material is removed and the structure material can transition into a solid or semi-solid state and thereby resisting deformation while in the material deposition region 106.

The structure material can comprise a polymer, such as, for example, a hydrogel, a thermoset polymer, a thermoplastic, or a combination thereof. The polymer can comprise a polymeric resin (e.g., a pre-polymer resin), a curing agent, a contrast agent, and/or other additives. For example, the polymer can comprise a collagen material, an alginate material, a decellularized extracellular matrix material, a fibrinogen material, a fibrin material, a hyaluronic acid material, a protein material, a polysaccharide hydrogel material, a synthetic gel material, an elastomeric polymer material, a rigid polymer material, a Matrigel, or a combination thereof. In various examples, the polymer can comprise a collagen material. The structure material can comprise a decellularized extracellular matrix. The structure material can comprise a concentration of collagen of at least 1 mg per mL structure material, such as, for example, at least 5 mg per mL of structure material, at least 10 mg per mL of structure material, at least 15 mg per mL of structure material, or at least 20 mg per mL of structure material. In certain examples, the structure material can comprise a concentration of collagen in a range of 1 mg per mL of structure material to 250 mg per mL of structure material, such as, for example, 10 mg per mL of structure material to 200 mg per mL of structure material. In various examples, the structure material comprises a fluid that transitions to a solid or semi-solid state after deposition.

The structure material can be dissolvable, such as, for example, an enzymatically dissolvable material (e.g., an alginate material, a collagen material).

The material deposition region 106 can be configured for mechanically supporting and/or holding the support material 108 during FRE additive manufacturing. For example, the material deposition region 106 can comprise a vessel in which the support material 108 is disposed and a platform on which the vessel is supported. The material deposition region 106 can comprise a motor and/or actuator that can move the platform in 3D space as needed.

The support material 108 can physically support at least a portion of the embedded bioink (i.e., object 114) and/or embedded structure material (e.g., scaffold 118), or a combination thereof. The support material 108 maintains the intended geometry of the embedded bioink and/or embedded structure material, and inhibits deformation of the respective material during the FRE additive manufacturing process. For example, the embedded bioink and/or embedded structure material can be held in position within the support material 108 until the bioink and/or structure material is cultured, solidified, and/or cured. The support material 108 can be stationary at an applied stress level below a threshold stress level and can flow at an applied stress level at or above the threshold stress level during the FRE additive manufacturing process.

The support material 108 can be a viscoplastic material with Bingham plastic-like rheological behavior. The support material 108 may demonstrate a significant shear thinning behavior such that the support material 108 acts like a solid material during deposition of the structure material and bioink, and then acts like a fluid when the nozzle 110a and/or nozzle 110b is moved through the support material 108 such that the movement of the nozzle 110a and/or nozzle 110b does not disturb the previously deposited structure material and/or bioink. A decrease in viscosity of the support material 108 under shear stress can make the support material 108 suitable for FRE. For example, in FRE, the dynamic loading can be caused by the force of the nozzle 110a and/or nozzle 110b through the support material 108, affecting the support material 108 in a number of ways. The extruder assembly 102a and/or extruder assembly 102b can be configured to change the support material 108 by imposing a mechanical load via shear, pressure, or vibration. The extruder assembly 102a and/or extruder assembly 102b can be configured to irradiate or heat the support material 108 to thin the support material 108. In various examples, the support material 108 can reduce viscosity under vibration, heating, or irradiation that occurs locally to the extruder assembly 102a and/or extruder assembly 102b, as the case may be.

The support material 108 can comprise other materials with viscoplastic behavior, such as Herschel-Bulkley fluid. Bingham plastics and Herschel-Bulkley fluids are viscoplastic materials included in the “shear-thinning” or “yield-stress fluid” category. Below a specific shear stress, these materials appear as a solid material. Above a threshold shear force, these materials behave as a fluid. A Bingham plastic may not necessarily “shear thin,” but rather may act much like a Newtonian fluid once it begins to flow. In contrast, the Herschel-Buckley fluid undergoes shear thinning once it begins to flow.

In various examples, the bioink, the structure material, and/or the support material can comprise microspheres. For example, microspheres in the bioink can comprise a mean average particle size (e.g., D₅₀) in a range of 50 microns to 2 mm. Microspheres in the support material and/or the structure material can comprise a mean average particle size in a range of 1 micron to 250 microns.

The support material can comprise a hydrogel. The hydrogel can comprise particles (e.g., microparticles) in a diluent. The particles can comprise gelatin or other suitable particle forming compound. The diluent can be aqueous or non-aqueous depending on the desired properties of the support material. Depending on the printing technique, the support material can be clear or opaque. In various examples, the support material can comprise thrombin, which may cure a respective material by cleaving fibrinogen into fibrin. The bioink and/or structure material can begin to cure when it contacts the support material. The support material can comprise a concentration of thrombin of at least 0.01 unit (U)/mL of support material, such as, for example, at least 0.05 U/mL of thrombin, or at least 0.1 U/mL. In certain examples, the support material can comprise a concentration of thrombin in a range of 0.01 U/mL to 10 U/mL, such as, for example, 0.05 U/mL to 1 U/mL.

The structure material and/or bioink can be curable and after curing, the structure material and/or bioink can be considered cured. The object 114 can be at least partially cured in the support material 108 after deposition of the bioink and the scaffold 118 can be at least partially cured in the support material 108 after deposition of the structure material. In various examples, the object 114 and scaffold 118 can be at least partially cured prior to removing the support material 108. In some examples, the object 114 and scaffold 118 may not be cured until after removing the support material 108. As used in this specification, the terms “cure” and “curing” can refer to the chemical crosslinking of components in the structure material. Accordingly, the terms “cure” and “curing” do not encompass solely physical drying of structure material through solvent or carrier evaporation. In this regard, the term “cured,” as used in this specification, refers to the condition of the structure material in which a component of the structure material forming the scaffold 118 or the bioink forming the object 114 has chemically reacted to form new covalent bonds in the structure material and/or bioink (e.g., new covalent bonds formed between a polymeric resin and a curing agent), new ionic bonds, new hydrogen bonds, new Vander walls bonds, or combinations thereof.

For example, curing of the object 114 and scaffold 118 can comprise cross-linking. The object 114 and scaffold 118 can be treated through various cross-linking techniques to selectively increase the rigidity of the overall object 114, scaffold 118, or portions thereof. Cross-linking can be induced by various mechanisms such as, for example, photo mechanisms (e.g., exposing the structure material to UV light), ionic mechanism, enzymatic mechanism, pH mechanisms (e.g., exposing the structure material to a different pH) or thermally driven mechanisms (e.g., cooling, heating). In various examples, the support material 108 can include a cross-linking agent or pH suitable for curing the structure material and/or bioink as it is deposited into the support material 108.

The mechanical properties of the object 114 and/or scaffold 118 can be controlled by controlling the amount of curing that occurs within the respective object 114 or scaffold 118. For example, the machine pathing instructions 132 can be modified to control the amount of crosslinking that occurs within the object 114 and/or scaffold 118. For example, the extruder assembly 102a, the extruder assembly 102b, and/or other assembly can comprise a UV light and can selectively subject the embedded structure material to the UV light as desired.

The object 114 and scaffold 118 can be at least partially removed from the support material 108. Removing the support material 108 may include heating the support material 108, cooling the support material 108, removing cations to disrupt crosslinking of the support material 108, physically removing the support material 108, vibration, irradiation with ultraviolet, infrared, or visible light, application of a constant or oscillating electric or magnetic field, other mechanism, or a combination thereof. For example, the support material can comprise a thermoreversible material and removing the support material can comprise heating the support material to a threshold temperature at which the support material transitions from a solid or semi-solid state to a liquid state.

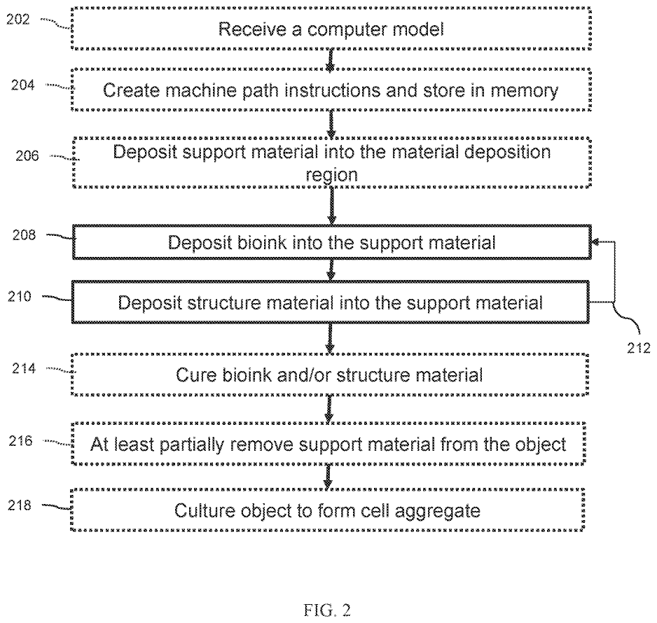

The methods for additive manufacturing herein, such as those illustrated in described in FIG. 2 below, can be implemented in whole or in part as computer-executable instructions stored in the memory 122 of the computer system 104 that, when executed by a processor 120 of the computer system 104, cause the computer system 104 to perform the enumerated steps. The computer instructions can be implemented as one or more software modules 116 stored in the memory 122 that are each programmed to cause the processor 120 to execute one or more discrete steps of the processes described herein or other functions. For example, the software modules 116 can comprise a separation module programmed to convert the computer model 124 into segments; a conversion module programmed to convert the computer model 124 and/or segments into computer instructions (e.g., G-code) for controlling the movement of the extruder assembly 102a and/or extruder assembly 102b to fabricate the object 114 and scaffold 118; an imaging module for controlling imaging parameters; a modeling module programmed to receive, store, create, and/or modify part files of objects to be fabricated; and a robotic control module programmed to control the extruder assembly 102a and/or extruder assembly 102b according to the instructions generated by the conversion module to fabricate the object 114 and scaffold 118. Various other modules can be implemented in addition to or in lieu of the aforementioned modules. In certain examples, the processes described herein can be executed across multiple computer systems that are communicably connected together in a network, a computer system communicably connected to a cloud computing system configured to execute one or more of the described steps, and so on.

Referring to FIG. 2, a flow chart illustrating an additive manufacturing method according to certain implementations of the present disclosure is provided. The method comprises receiving, by the processor 120, a computer model 124 of the assembly at step 202. At step 204, the processor 120, executing the separation module software, can separate (e.g., slice) the computer model into different part segments and the processor 120, executing the conversion module, can create machine path instructions (e.g., G-code instructions) based on the design computer model. The machine path instructions can be stored in memory 122. The method can optionally comprise depositing the support material in the material deposition region prior to printing of the object at step 206.

The method can comprise, at step 208, depositing bioink into the support material based on the computer model 124 of the assembly, thereby forming a portion 114a of the object 114 in the support material 108. Depositing the bioink into the support material 108 can be based on the machine path instructions for the computer model 124.

The method can comprise, at step 210, depositing structure material into the support material based on the computer model 124 of the assembly, thereby forming a portion 118a of the scaffold 118 for the object 114 in the support material 108. Depositing the structure material into the support material 108 can be based on the machine path instructions for the computer model 124. The machine path instructions can be configured to avoid passing over the scaffold 118 while depositing the bioink.

The depositing of the bioink and/or structure material can be repeated over as many iterations as necessary to additively form the object 114 as shown by feedback loop step 212. Each iteration can deposit portions of the bioink and structure material (steps 208 and 210) and the iterations can be repeated until additive formation of the object 114 and scaffold 118 is complete (if not aborted earlier). In various examples, an additional bioink and/or an additional structure material can be deposited as necessary to form the object 114 and/or scaffold 118 such that at least two bioinks are deposited at step 208 and/or at least two structure materials are deposited at step 210.

Thereafter, at step 214, the structure material and/or bioink can be at least partially cured after depositing and then, at step 216, the support material can be at least partially removed from the object 114. The curing can occur prior to, during, after, or a combination thereof, removal of the support material at step 216.

At step 218, the object 114 can be cultured to form a cell aggregate. Culturing is a process under which cells are grown under desirable conditions. For example, the bioink can be maintained at a temperature proximal to normal human body temperature (e.g., 37° C.+/−2° C.), maintained with humidity, maintained with suitable carbon dioxide levels, and optionally supplied with suitable nutrients and/or additives to facilitate cell growth. Culturing can result in compaction of the object 114 around the scaffold 118 to form a cell aggregate. As used herein, “compaction” means a deformation of an element, which can include a reduction in size (e.g., volume change) and/or a change in shape (e.g., bend, twist), but is not necessarily limited thereto.

The cell aggregate can comprise an organoid, a spheroid, or a combination thereof. The organoid can exhibit a functionality of a tissue or an organ. The organoid can comprise an intestinal organoid, a stomach organoid, a liver organoid, a kidney organoid, a cardiac organoid, a muscle organoid, a brain organoid, other tissue type, or a combination of any thereof. The organoid can be used for basic science, disease modeling, drug development, cell therapy, tissue engineering, regenerative medicine applications, or a combination thereof.

The cell aggregate can comprise a diameter in a range of 100 μm to 1 mm. The object 114, prior to compaction, can comprises a diameter in a range of 1.1 mm to 2 cm, such as, for example, 1.1 mm to 1 cm, 1.1 mm to 100 mm, or 1.1 mm to 10 mm. During compaction, the object 114 can decrease in volume by at least 10% by volume, at least 20% by volume, at least 30% by volume, at least 40% by volume, at least 50% by volume, at least 60% by volume, at least 70% by volume, at least 80% by volume, or at least 90% by volume.

Cells in non-natural environments often lack the structural cues to organize within an organoid in the same manner as in native tissue. For example, native cardiac tissue and/or other muscular tissue may have formed aligned, elongated structures suitable for desirable transport of nutrients, oxygen, and/or metabolic wastes as well as muscle contraction. Cells in non-natural environments may lack the structural cues necessary to form the desired structures and may form shapes (e.g., spheres), which may be undesirable for nutrient, oxygen, and/or metabolic waste transport without the use of a vasculature-like transportation system.

The scaffold 118 can guide the compaction of the object 114, such as, for example, to a desired shape similar to the shape of the native tissue and/or organ. The shape and/or size of the scaffold 118 and/or contact between the scaffold 118 and the object 114 can influence the shape of the object 114 after compaction. The scaffold 118 can be internal to the object 114. The scaffold 118 can provide structural cues to the cells in the object 114 to aid in cellular organization and determination of cell construct's the terminal structure. Cells can be patterned around the scaffold 118 so that when they begin to establish cell to cell junctions and pull themselves together, they also begin to interact with the internal scaffold. The scaffold 118 can provide resistance to the compaction process as the cells pull each other together. This resistive force can prevent the cells from pulling themselves completely together into an amorphous spheroid of cells which guides the cells into their postcompaction, or terminal, shape.

The cells can establish an axis or axes of tension as they pull against the scaffold 118, providing the scaffold is in an anisotropic shape. This axis or axes provide organizational cues to the cells in which they will begin to align themselves along.

For example, the scaffold 118 can be biocompatible and interact with the cells in the object 114. The bioink in the object 114 can maintain a printed geometry of the cells until the cells establish cell to cell junctions. The cells can also degrade the fibrinogen in the bioink. As the object 114 compacts and the cells pull themselves together they can bind to the scaffold 118.

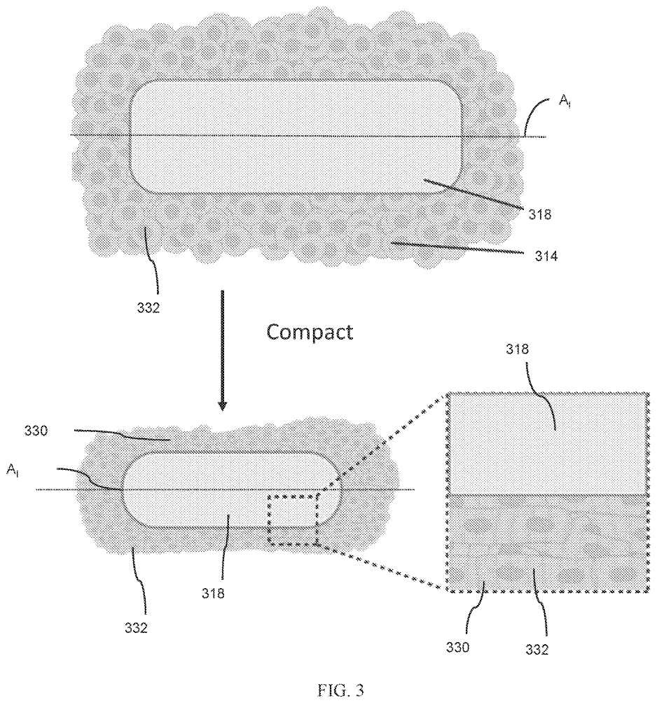

For example, compaction of an object 314 formed form bioink and a scaffold 318 formed from structure material is illustrated in FIG. 3. The object 314 after culturing formed cell aggregate 330 and the cells can bind to the scaffold 318. As illustrated, the cells 332 have substantially formed a desirable shape and alignment. For example, the cells 332 in the cell aggregate 330 are elongated (e.g., increased in aspect ratio (length: width ratio) compared to the cells 332 in the object 314. The cells 332 in the cells aggregate 332 can be substantially aligned with a longitudinal axis, A₁ of the scaffold 318. In various examples, the cell aggregate 330 can be a cardiac organoid, a muscle organoid, or a combination thereof and the cell aggregate 330 can comprise an increased twitch force, a faster conduction velocity, or a combination thereof compared to a conventional cell aggregate formed without the scaffold 318. A conventional cell aggregate formed without the scaffold 318 may not formed elongated cells aligned with the longitudinal axis, A₁ of the scaffold 318. The alignment of the cells 332 can influence the function of the cells 332. In certain examples, the cells 332 can comprise stem cells that may differentiate based on the alignment, such that the mechanical tensions within the cells 332 can change the gene expression within the cells 332 and subsequent cell-mediated processes.

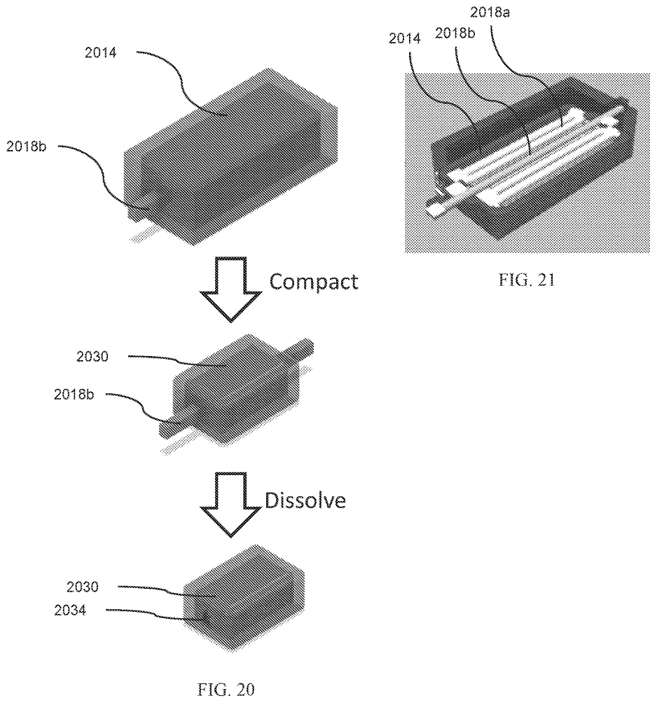

In various examples, an additional bioink and/or an additional structure material may be deposited. For example, as illustrated in FIG. 20 and a cross sectional view in FIG. 21, an object 2014 has been printed with a scaffold 2018a and a scaffold 2018b. The structure material in each scaffold 2018a, 2018b can the same or different. As illustrated, the scaffold 2018a and scaffold 2018b are different. For example, the scaffold 2018a can comprise a collagen material and the scaffold 2018b can comprise an alginate material.

After printing the object 2014 can compact to form cell aggregate 2030. Then, the scaffold 2018b can be dissolve with, for example, an enzyme thereby forming a bore 2034 through the object 2014. The scaffold 2018a may remain intact.

The methods for additive manufacturing and systems for additive manufacturing described herein can be used to create various products. The products can be various product types, such as, for example, a soft structure, a bioprosthetic, a scaffold, a medical device, an implantable device, a gasket, a tube, a seal, an aerospace part, an automotive part, a building component, or other structures that may be additively manufactured. In various examples, the product (e.g., object 114) can be surgically fit into a patient after additive manufacturing, the object 114 can be utilized as a biological structure for experimentation, or a combination thereof.

The present disclosure can provide a precise control of deposition of bioink, thereby the number of cells in a cell aggregate formed therefrom can be controlled. Controlling the number of cells and/or the shape into which the cells will form, can result in reproducible cells aggregates that can exhibit consistent behavior and a consistent final form.

### EXAMPLES

Various aspects, benefits and features that are potentially realizable through implementation of the present invention will be more fully understood by reference to the following examples, which provide illustrative non-limiting aspects of the invention. It is understood that the invention described in this specification is not necessarily limited to the examples described in this section.

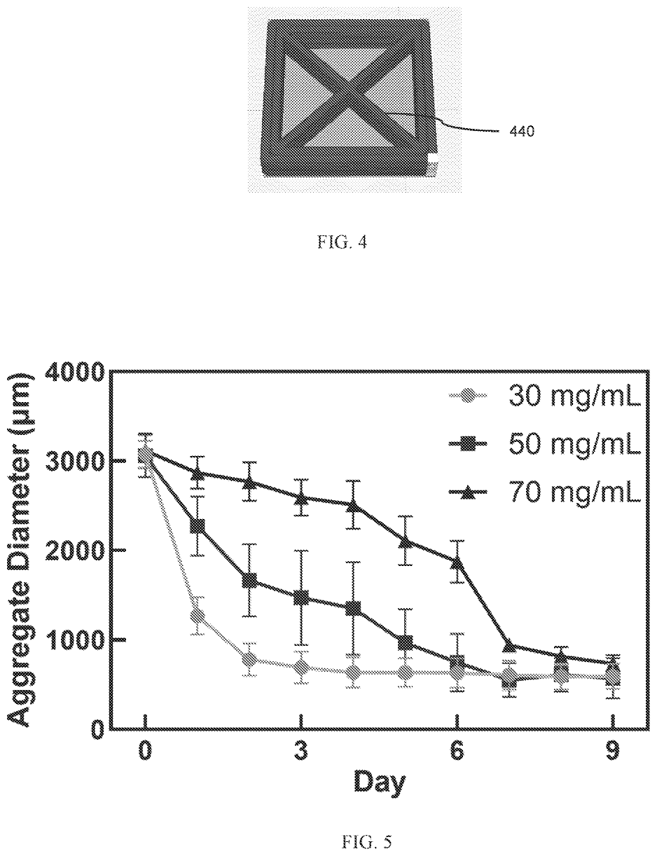

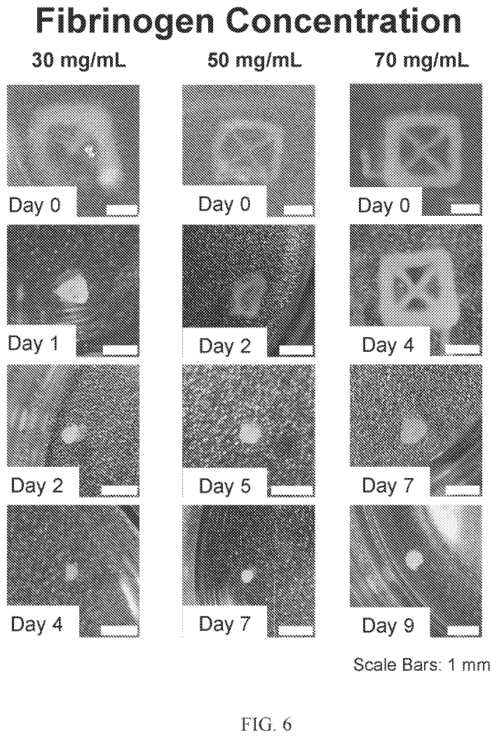

A computer model was created for an object as shown in FIG. 4. The object was additively manufactured into a support material solely with a bioink comprising cells and fibrinogen. No structure material was used. In a first experiment, the object shown in FIG. 4 was additively manufactured various times while using bioinks with different fibrinogen concentrations (30 mg/mL, 50 mg/mL, 70 mg/mL) while maintaining the same cell concentration in the bioink. The objects were cultured and compacted as shown in FIG. 6 and the mean average diameter of the objects over time was measured as shown in FIG. 5. While the increase in fibrinogen decreased the initial compaction rate, the final size of the cell aggregate formed from each object was similar and the shapes were similar (e.g., spherical). The higher fibrinogen concentration print was observed to have a higher print fidelity than the lower fibrinogen concentration prints. The internal ribs 440 shown in FIG. 4 were observed to minimally, if at all, affect the final shape of the cell aggregate.

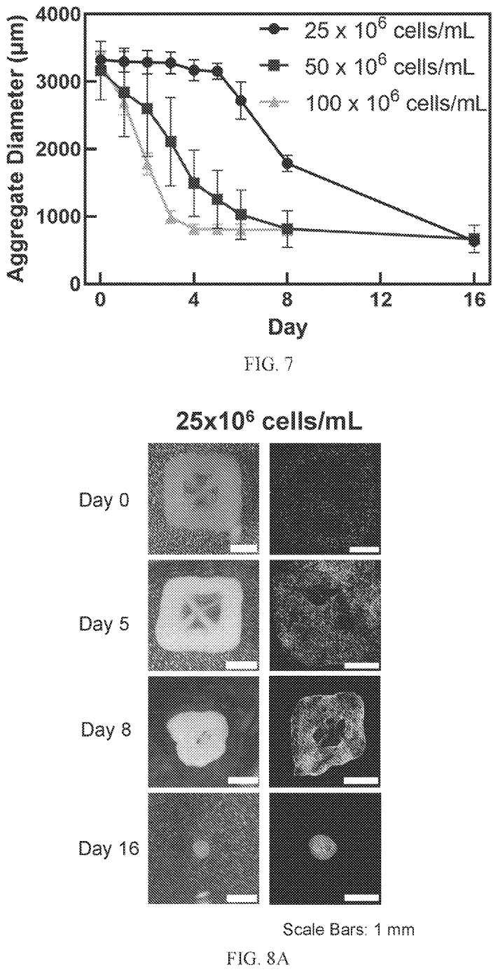

In a second experiment, the object was additive manufactured various times into a support material multiple times while using bioinks with different cell concentrations (25 million cells/mL, 50 million cells/mL, 100 million cells/mL) while maintaining the same fibrinogen concentration. The objects were cultured and compacted as shown in FIGS. 8A-8B and the mean average diameter of the objects over time was measured as shown in FIG. 7. While the increase in cell concentration increased the initial compaction rate, the final size of the cell aggregate formed from each object was similar and the shapes were similar (e.g., spherical). The cell concentration was observed to have a minimal effect on print fidelity.

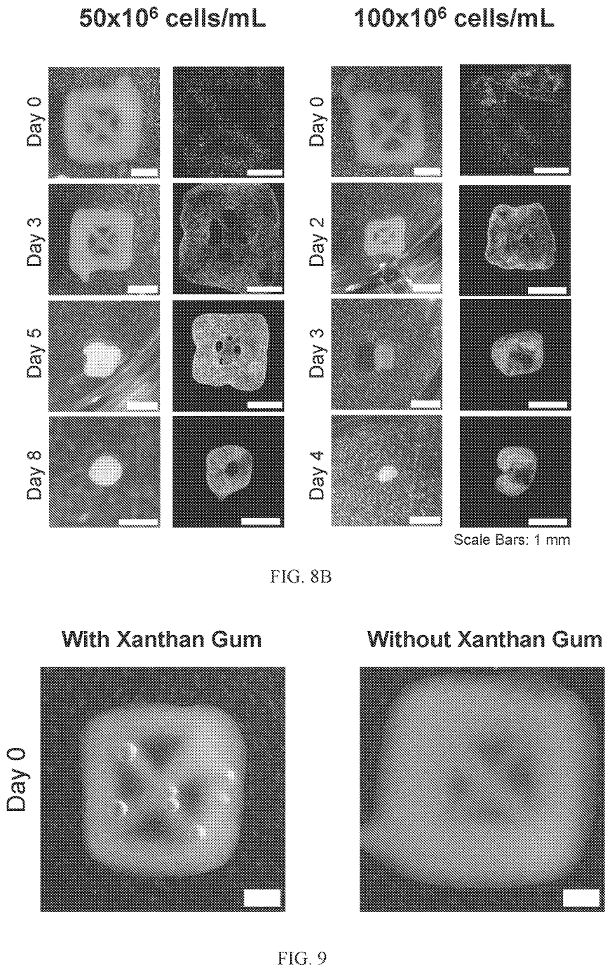

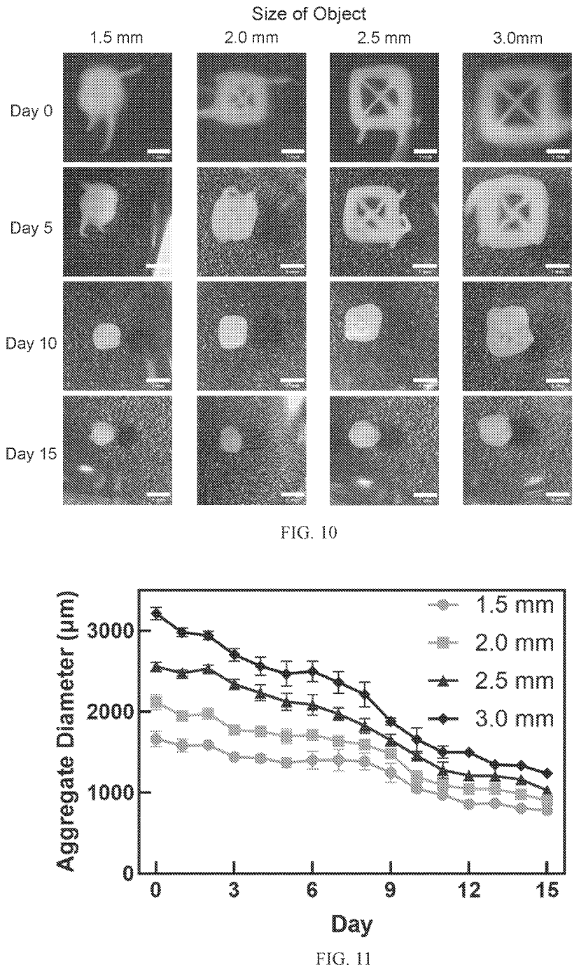

In a third experiment, the object was additively manufactured twice into a support material using bioinks with the same cell concentration and same fibrinogen concentrations, but one contained xanthan gum and one did not contain xanthan gum. The initial results of the printing showed enhanced print quality with the xanthan gum as illustrated in FIG. 9.

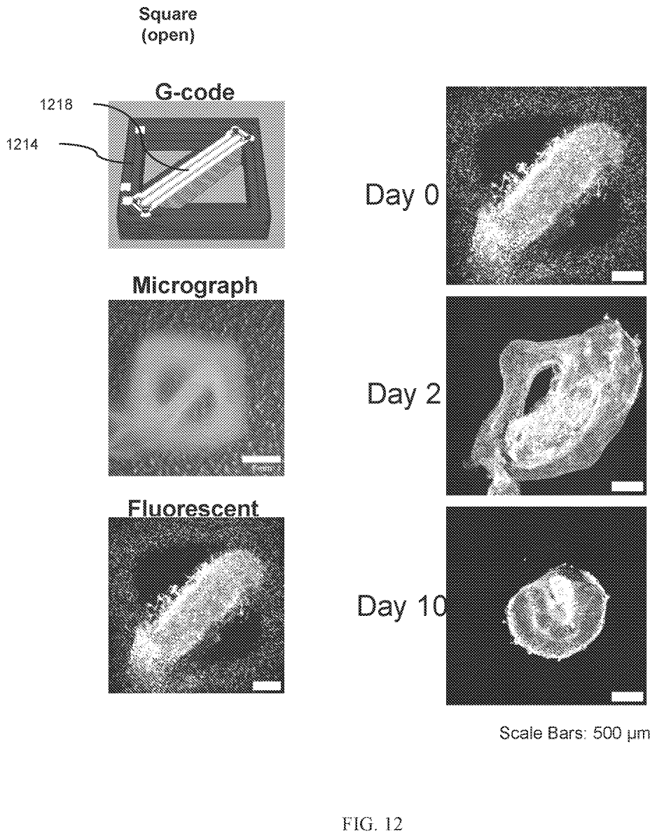

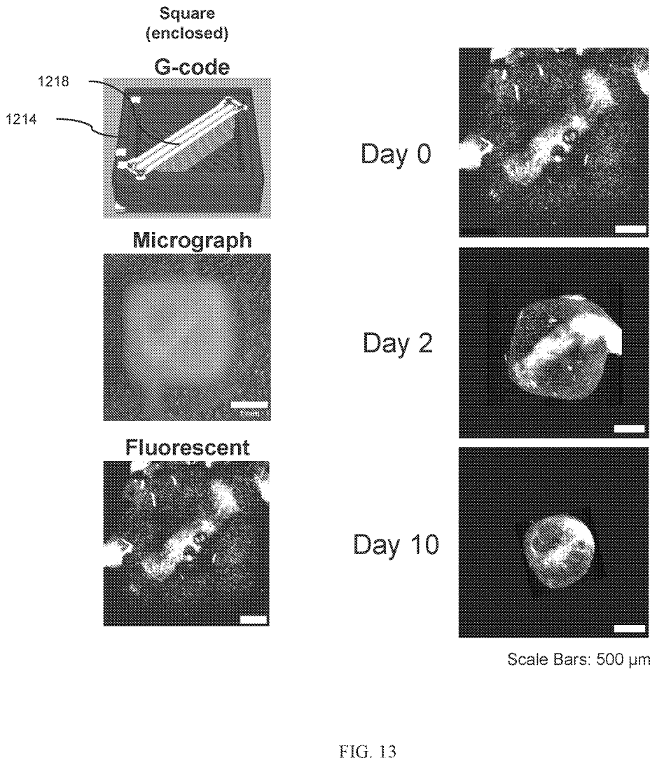

In a fourth experiment, the object was additively manufactured various times at various size scales (1.5 millimeter (mm) length, 2.0 mm length, 2.5 mm length, and 3.0 mm length) in a support material while maintaining the same composition of the bioink. The objects were cultured and compacted as shown in FIG. 10 and the mean average diameter of the objects over time was measured as shown in FIG. 11. It was observed that the initial additively manufactured size did not greatly affect compaction rate. It was also observed that objects that are initially additively manufactured larger, have a slightly larger size after compaction. It was observed that the scaffold being enclosed by cells to remain on the inside of the cell aggregate throughout the compaction process can enhance the alignment, shape, and/or size of the final cell aggregate.

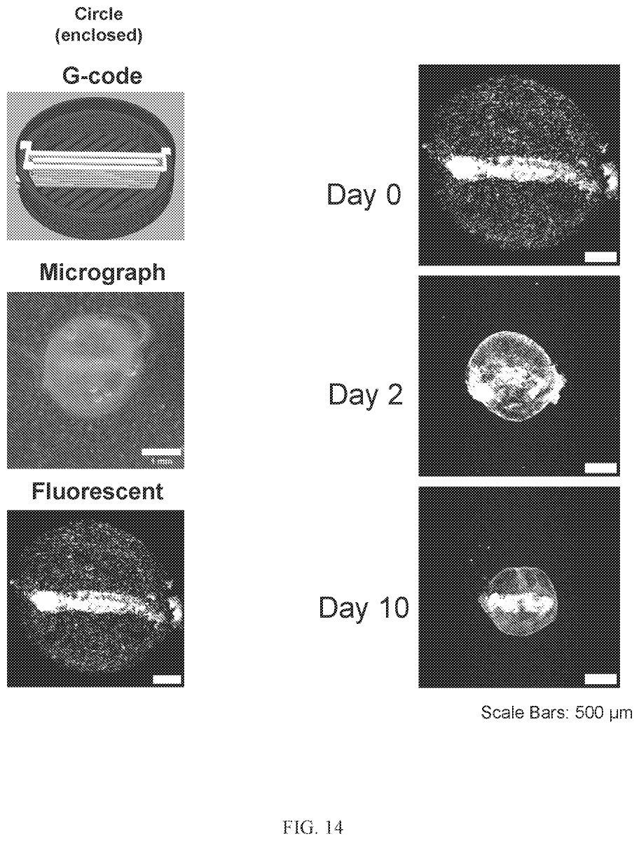

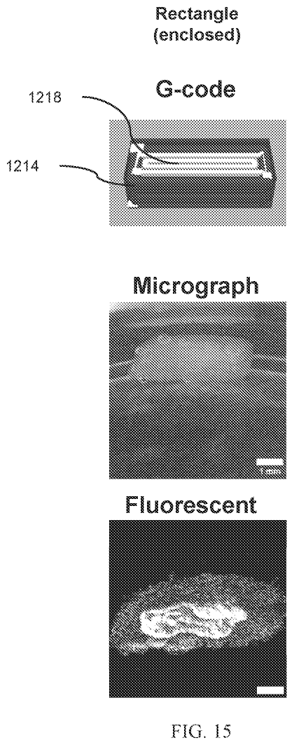

In a fifth experiment, four different objects (open square, enclosed square, enclosed circle, and enclosed rectangle) were additively manufactured into a support material with the same composition of the bioink comprising cells, fibrinogen, and xanthan gum, and the same composition of a structure material comprising collagen. As illustrated in FIGS. 12-15, the object 1214 was additively manufactured with bioink and the scaffold 1218 was additively manufactured with the structure material. The objects were cultured and compacted as shown in FIGS. 12-14. Each object was successfully printed and the shapes of the final cell aggregate were determined to have been desirable and guided by the respective scaffold 1218.

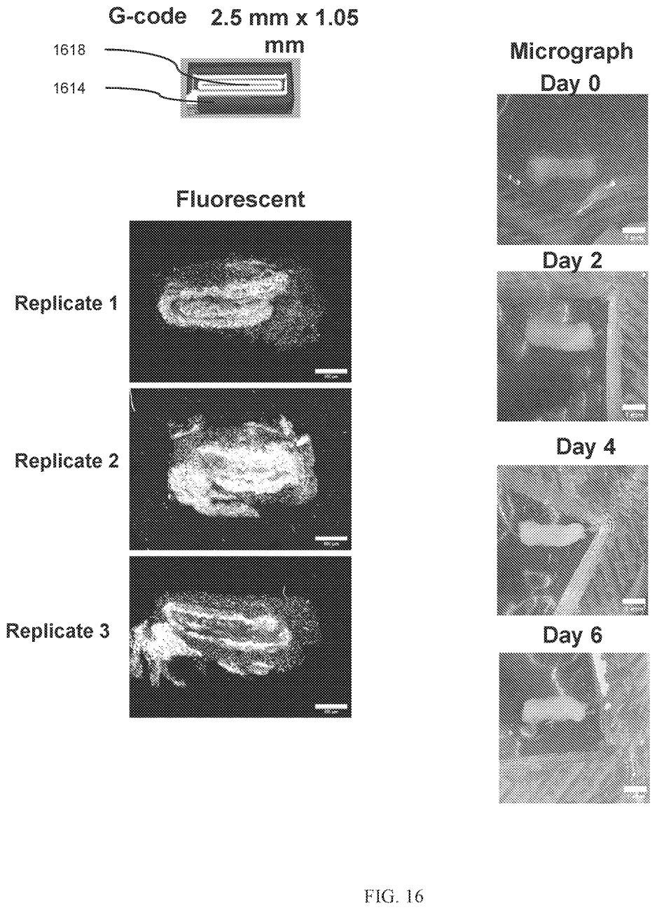

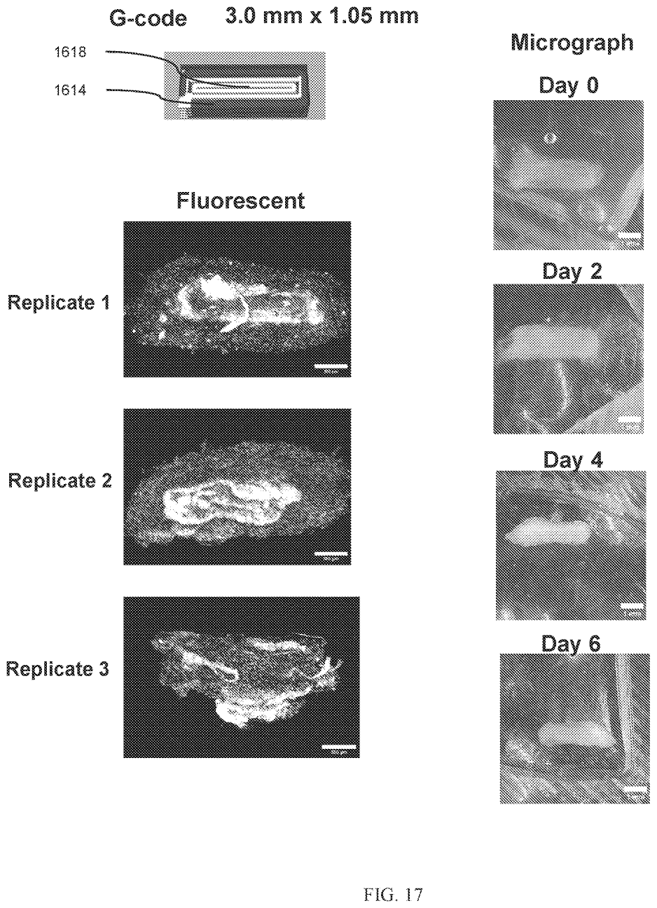

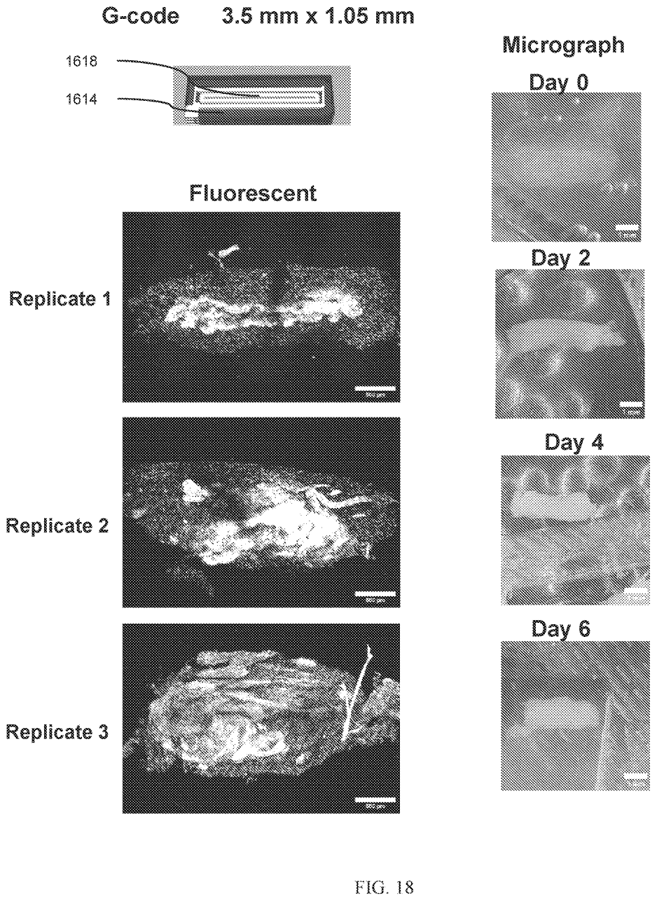

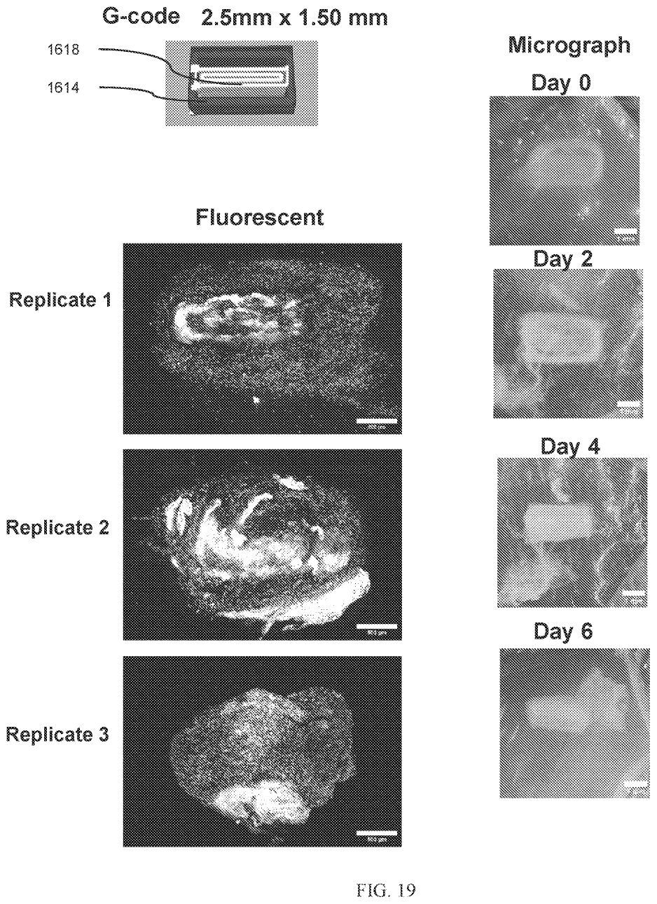

In a sixth experiment, four rectangular objects were additively manufactured into a support material with different sizes (2.5 mm×1.05 mm, 3.0 mm×1.05 mm, 3.5 mm×1.05 mm, 2.5 mm×1.50 mm) using a bioink comprising 70 mg/mL of fibrinogen, 1% xanthan gum by weight, and 100 million C2C12 cells (mouse myoblasts) per mL. The structure material comprised collagen and the support material comprised 0.1 U/mL of thrombin. As illustrated in FIGS. 16-19, each object 1614 was printed with a corresponding scaffold 1618. The objects 1614 were cultured and compacted as shown in FIG. 16-19. Each object was successfully printed and the shapes of the final cell aggregate were determined to have been desirable and guided by the respective scaffold 1618.

Various aspects of non-limiting embodiments of an invention according to the present disclosure include, but are not limited to, the aspects listed in the following numbered clauses.

Clause 1. An additive manufacturing method comprising: depositing a bioink into a support material based on a first computer model of an object and a scaffold for the object, thereby forming a first portion of the object in the support material, wherein the bioink comprises cells; depositing a structure material into the support material based on the first computer model, thereby forming a first portion of the scaffold, wherein the structure material is different from the bioink and the structure material comprises a polymer; and repeating the depositing of the bioink and the structure material as necessary to additively form the object and the scaffold within the object.

Clause 2. The method of clause 1, wherein the bioink further comprises a bio compatible polymer and water.

Clause 3. The method of clause 2, wherein the bio compatible polymer is fibrinogen and the bioink comprises at least 30 milligrams of fibrinogen per milliliter of bioink.

Clause 4. The method of clause 3, wherein the bioink comprises at least 50 milligrams of fibrinogen per milliliter of bioink.

Clause 5. The method of any of clauses 1-4, wherein the bioink comprises at least 25 million cells per milliliter of bioink.

Clause 6. The method of any of clauses 1-5, wherein the cells are eukaryotic cells derived from an animal.

Clause 7. The method of any of clauses 1-6, wherein the bioink comprises a rheological modifier.

Clause 8. The method of any of clauses 1-7, wherein the structure material is acellular.

Clause 9. The method of any of clauses 1-8, wherein the bioink and the structure material differ by at least one mechanical property.

Clause 10. The method of clause 9, wherein the mechanical property is a yield strength, a stiffness, a tensile strength, or a combination thereof.

Clause 11. The method of any of clauses 1-10, wherein the polymer of the structure material comprises a hydrogel, a thermoset polymer, a thermoplastic polymer, or a combination thereof.

Clause 12. The method of any of clauses 1-11, wherein the polymer comprises a collagen material, an alginate material, a decellularized extracellular matrix material, a fibrinogen material, a fibrin material, a hyaluronic acid material, a protein material, a polysaccharide hydrogel material, a synthetic gel material, an elastomeric polymer material, a rigid polymer material, a Matrigel, or a combination thereof.

Clause 13. The method of any of clauses 1-12, wherein the scaffold is configured to guide the compaction of the object.

Clause 14. The method of any of clauses 1-13, wherein the support material is configured to physically support the bioink, the structure material, or a combination thereof during deposition and the support material is stationary at an applied stress level below a threshold shear stress level and flows at an applied shear stress level at or above the threshold shear stress level.

Clause 15. The method of any of clauses 1-14, wherein the support material comprises a hydrogel.

Clause 16. The method of clause 15, wherein the bioink further comprises fibrinogen and water, the polymer comprises a collagen material, the structure material is acellular, and the support material comprises thrombin.

Clause 17. The method of any of clauses 1-16, further comprising culturing the object to form a cell aggregate.

Clause 18. The method of clause 17, wherein culturing results in compaction of the object around the scaffold to form the cell aggregate.

Clause 19. The method of clause 18, wherein the cell aggregate comprises an organoid, a spheroid, or a combination thereof.

Clause 20. The method of any of clauses 18-19, wherein the cell aggregate comprises elongated cells that are substantially aligned with a longitudinal axis of the scaffold.

Clause 21. The method of clause 20, wherein the cell aggregate is a cardiac organoid, a muscle organoid, or a combination thereof.

Clause 22. The method of clause 21, wherein the cell aggregate has an increased twitch force, a faster conduction velocity, or a combination thereof compared to a conventional cell aggregated formed without the scaffold.

Clause 23. The method of any of clauses 17-22, wherein the object decreases by at least 10% by volume to form the cell aggregate.

Clause 24. The method of any of clauses 17-23, further comprising surgically fitting the cell aggregate into a patient, utilizing the object as a biological structure for experimentation, or a combination thereof.

Clause 25. The method of any of clauses 17-24, further comprising creating machine path instructions for the first computer model, wherein depositing the structure material into the support material is based on the machine path instructions for the first computer model.

Clause 26. The method of clause 25, wherein the machine path instructions avoid passing over the scaffold while depositing the bioink.

Clause 27. The method of any of clauses 25-26, wherein the first computer model is generated from image data of a biological structure, an engineered structure, a computationally derived structure, or a combination thereof.

Clause 28. The method of any of clauses 1-27, further comprising, after depositing, at least partially curing the bioink, the structure material, or a combination thereof.

Clause 29. The method any of clauses 1-28, further comprising at least partially removing the support material.

Clause 30. The method of clause 29, wherein: the support material comprises a thermoreversible material; and at least partially removing the support material comprises heating the support material to a threshold temperature at which the support material transitions from a solid or semi-solid state to a liquid state.

Clause 31. An additive manufacturing system comprising: an extruder assembly comprising a first nozzle and a second nozzle, wherein the first nozzle is configured to deposit, according to a first computer model, bioink into a support material to additively form an object made of the bioink in the support material, and the second nozzle is configured to deposit, according to the first computer model, structure material into a support material to additively form a scaffold made of the structure material in the support material; a material deposition region configured to hold the support material; and a processor that is in communication with the extruder assembly, wherein the processor is programmed to perform the method of any of clauses 1-30.

Clause 32. A product fabricated by the method of any of clauses 1-30 or the additive manufacturing system of clause 31.

Clause 33. The product of clause 32, wherein the product comprises elongated cells that are substantially aligned with a longitudinal axis of the scaffold.

Certain exemplary aspects of the present disclosure will now be described to provide an overall understanding of the principles of the structure, function, manufacture, and use of the compositions, methods, and products disclosed herein. One or more examples of these aspects are illustrated in the accompanying drawings. Those of ordinary skill in the art will understand that the devices and methods specifically described herein and illustrated in the accompanying drawings are non-limiting exemplary aspects and that the scope of the various examples of the present disclosure is defined solely by the claims. The features illustrated or described in connection with one exemplary aspect may be combined with the features of other aspects. Such modifications and variations are intended to be included within the scope of the present disclosure.

Any references herein to “various examples,” “some examples,” “one example,” “an example,” similar references to “aspects,” or the like, means that a particular feature, structure, or characteristic described in connection with the example is included in at least one example. Thus, appearances of the phrases “in various examples,” “in some examples,” “in one example,” “in an example,” similar references to “aspects,” or the like, in places throughout the specification are not necessarily all referring to the same example. Furthermore, the particular features, structures, or characteristics may be combined in any suitable manner in one or more examples. Thus, the particular features, structures, or characteristics illustrated or described in connection with one example may be combined, in whole or in part, with the features, structures, or characteristics of one or more other examples without limitation. Such modifications and variations are intended to be included within the scope of the present examples.

Any patent, publication, or other disclosure material identified herein is incorporated herein by reference in its entirety unless otherwise indicated but only to the extent that the incorporated material does not conflict with existing definitions, statements, or other disclosure material expressly set forth in this specification. As such, and to the extent necessary, the express disclosure as set forth in this specification supersedes any conflicting material incorporated by reference herein. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicant reserves the right to amend this specification to expressly recite any subject matter, or portion thereof, incorporated by reference herein.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

The grammatical articles “a,” “an,” and “the,” as used herein, are intended to include “at least one” or “one or more,” unless otherwise indicated, even if “at least one” or “one or more” is expressly used in certain instances. Thus, the articles are used herein to refer to one or more than one (i.e., to “at least one”) of the grammatical objects of the article. Further, the use of a singular noun includes the plural, and the use of a plural noun includes the singular, unless the context of the usage requires otherwise.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

One skilled in the art will recognize that the herein described articles and methods, and the discussion accompanying them, are used as examples for the sake of conceptual clarity and that various configuration modifications are contemplated. Consequently, as used herein, the specific examples/embodiments set forth and the accompanying discussions are intended to be representative of their more general classes. In general, use of any specific exemplar is intended to be representative of its class, and the non-inclusion of specific components, devices, operations/actions, and objects should not be taken to be limiting. While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed and not as more narrowly defined by particular illustrative aspects provided herein.

What is claimed is:

1. An additive manufacturing method comprising:
depositing a bioink into a support material based on a first computer model of an object and a scaffold for the object, thereby forming a first portion of the object in the support material, wherein the bioink comprises cells;
depositing a structure material into the support material based on the first computer model, thereby forming a first portion of the scaffold, wherein the structure material is different from the bioink and the structure material comprises a polymer; and
repeating the depositing of the bioink and the structure material as necessary to additively form the object and the scaffold within the object.

2. The method of claim 1, wherein the bioink further comprises a bio compatible polymer and water.

3. The method of claim 2, wherein the bio compatible polymer is fibrinogen and the bioink comprises at least 30 milligrams of fibrinogen per milliliter of bioink.

4. The method of claim 3, wherein the bioink comprises at least 50 milligrams of fibrinogen per milliliter of bioink.

5. The method of claim 1, wherein the bioink comprises at least 25 million cells per milliliter of bioink.

6. The method of claim 1, wherein the cells are eukaryotic cells derived from an animal.

7. The method of claim 1, wherein the bioink comprises a rheological modifier.

8. The method of claim 1, wherein the structure material is acellular.

9. The method of claim 1, wherein the bioink and the structure material differ by at least one mechanical property.

10. The method of claim 9, wherein the mechanical property is a yield strength, a stiffness, a tensile strength, or a combination thereof.

11. The method of claim 1, wherein the polymer of the structure material comprises a hydrogel, a thermoset polymer, a thermoplastic polymer, or a combination thereof.

12. The method of claim 1, wherein the polymer comprises a collagen material, an alginate material, a decellularized extracellular matrix material, a fibrinogen material, a fibrin material, a hyaluronic acid material, a protein material, a polysaccharide hydrogel material, a synthetic gel material, an elastomeric polymer material, a rigid polymer material, a Matrigel, or a combination thereof.

13. The method of claim 1, wherein the scaffold is configured to guide compaction of the object.

14. The method of claim 1, wherein the support material is configured to physically support the bioink, the structure material, or a combination thereof during deposition and the support material is stationary at an applied stress level below a threshold shear stress level and flows at an applied shear stress level at or above the threshold shear stress level.

15. The method of claim 1, wherein the support material comprises a hydrogel.

16. The method of claim 15, wherein the bioink further comprises fibrinogen and water, the polymer comprises a collagen material, the structure material is acellular, and the support material comprises thrombin.

17. The method of claim 1, further comprising culturing the object to form a cell aggregate.

18. The method of claim 17, wherein culturing results in compaction of the object around the scaffold to form the cell aggregate.

19. The method of claim 18, wherein the cell aggregate comprises an organoid, a spheroid, or a combination thereof.

20. The method of claim 18, wherein the cell aggregate comprises elongated cells that are substantially aligned with a longitudinal axis of the scaffold.

21. The method of claim 20, wherein the cell aggregate is a cardiac organoid, a muscle organoid, or a combination thereof.

22. The method of claim 21, wherein the cell aggregate has an increased twitch force, a faster conduction velocity, or a combination thereof compared to a conventional cell aggregated formed without the scaffold.

23. The method of claim 17, wherein the object decreases by at least 10% by volume to form the cell aggregate.

24. The method of claim 17, further comprising surgically fitting the cell aggregate into a patient, utilizing the object as a biological structure for experimentation, or a combination thereof.

25. The method of claim 1, further comprising
creating machine path instructions for the first computer model, wherein depositing the structure material into the support material is based on the machine path instructions for the first computer model.

26. The method of claim 25, wherein the machine path instructions avoid passing over the scaffold while depositing the bioink.

27. The method of claim 25, wherein the first computer model is generated from image data of a biological structure, an engineered structure, a computationally derived structure, or a combination thereof.

28. The method of claim 1, further comprising, after depositing, at least partially curing the bioink, the structure material, or a combination thereof.

29. The method claim 1, further comprising at least partially removing the support material.

30. The method of claim 29, wherein:
the support material comprises a thermoreversible material; and
at least partially removing the support material comprises heating the support material to a threshold temperature at which the support material transitions from a solid or semi-solid state to a liquid state.

31. An additive manufacturing system comprising:
an extruder assembly comprising a first nozzle and a second nozzle, wherein
the first nozzle is configured to deposit, according to a first computer model, bioink into a support material to additively form an object made of the bioink in the support material, and
the second nozzle is configured to deposit, according to the first computer model, structure material into a support material to additively form a scaffold made of the structure material in the support material;
a material deposition region configured to hold the support material; and
a processor that is in communication with the extruder assembly, wherein the processor is programmed to perform the method of claim 1.

32. A product fabricated by the method of claim 1.

33. The product of claim 32, wherein the product comprises elongated cells that are substantially aligned with a longitudinal axis of the scaffold.
